(19) United States
(12) Patent Application Publication
(10) Pub. No.: US 2014/0235877 A1
(43) Pub. Date: Aug. 21, 2014

(54) COMPOUNDS, METHODS OF PREPARATION AND USE THEREOF FOR TREATING CANCER

(71) Applicant: Hong Kong Baptist University, Hong Kong (HK)

(72) Inventors: Hongxi XU, Hong Kong (HK); Zhengxiang XIA, Hong Kong (HK); Yuanzhi LAO, Hong Kong (HK); Hongsheng TAN, Hong Kong (HK); Zhaoxiang BIAN, Hong Kong (HK); Dajian YANG, Hong Kong (HK); Shilin CHEN, Hong Kong (HK); Kaixian CHEN, Hong Kong (HK); Albert Sun-Chi CHAN, Hong Kong (HK)

(73) Assignee: Hong Kong Baptist University, Hong Kong (HK)

(21) Appl. No.: 14/231,733

(22) Filed: Mar. 31, 2014

Related U.S. Application Data
(63) Continuation of application No. 13/767,885, filed on Feb. 15, 2013.

(51) Int. Cl. C07D 311/86; A61K 36/38

(52) U.S. Cl. CPC C07D 311/86; A61K 36/38
USPC 549/392

(57) ABSTRACT

The present invention provides two compounds, namely Compound A and B, as the potential anticancer drug, and a composition comprising said Compound A and/or B for treating cancer or tumor related diseases. The present invention also relates to methods of preparing the compounds from a natural source and a composition comprising the compounds, and using the same for treating cancer or tumor related diseases.

### CROSS-REFERENCE TO RELATED APPLICATIONS

The present application is a Continuation application of U.S. patent application Ser. No. 13/767,885 filed on Feb. 15, 2013, which claims priority of U.S. provisional applications No. 61/598,925 filed Feb. 15, 2012 and 61/641,914 filed May 3, 2012. All of the foregoing applications are hereby incorporated by reference in their entirety.

### FIELD OF INVENTION

The present invention is in the field of pharmaceuticals and chemical industries. In particular, the present invention relates to two new anti-cancer compounds, namely compound A and compound B, which are isolated from Garcinia nujiangensis. The present invention also includes the methods of preparation and use thereof for treating cancer or tumor related diseases.

### BACKGROUND OF INVENTION

In 2007, cancer caused about 13% of all human deaths worldwide (7.9 millions). Rates are rising as more people live to an old age and as mass lifestyle changes occur in the developing world. According to the Cancer Statistics published by the American Cancer Society, cancer is the second leading cause of mortality in the United States today.

Cancer management options include surgery, chemotherapy and radiation therapy etc. Complete removal of the cancer without damage to the rest of the body is the goal of treatment. Sometimes this can be accomplished by surgery, but the propensity of cancers to invade adjacent tissue or to spread to distant sites by microscopic metastasis often limits its effectiveness. The effectiveness of chemotherapy is often limited by toxicity to other tissues in the body. Radiation can also cause damage to normal tissue. Therefore, there is a large demand for developing novel compounds and methods that are more effective or less toxicity on cancer treatment.

The active natural products with novel structures are the important sources of lead compounds. The discovery of novel compounds will promote the development of chemistry and pharmaceutical research. Furthermore, it may also lead to the new anti-cancer drug development. Since over 60% chemotherapeutic drugs, such as paclitaxel, originate from plants, there is a growing interest in investigating of Traditional Chinese Medicine (TCM) herbs with anti-cancer activities to identify lead bioactive compounds as candidates for anticancer agents.

It is well known that the genus Garcinia of the family Guttiferae is a prolific source of polycyclic polyprenylated acylphloroglucinols and bioactive prenylated xanthones, which exhibit various biological activities including antibacterial, antifungal, anti-inflammatory, antioxidant, and cytotoxic effects. However, the anti-cancer potentials of most Garcinia species and their potential synergistic anti-cancer effects have not been investigated systematically.

U.S. Pat. No. 7,592,367 B2 and publication no. US 2011/0301233 A1 disclosed two other compounds derived from other genus Garcinia species that have therapeutic effects for treatment of cancers and tumors, but the compounds of the present invention have never been disclosed in the afore-mentioned patent or patent application or any references in the relevant art.

Citation or identification of any reference in this section or any other section of this application shall not be construed as an admission that such reference is available as prior art for the present application.

### SUMMARY OF INVENTION

Accordingly, the objective of the present invention is to provide two new compounds, namely Compound A and B, as the potential anticancer drug, and a composition comprising said Compound A and/or B for treating cancer or tumor related diseases.

In accordance with one aspect of the present invention, a method for extraction and separation of said compounds from their natural source is provided.

In another aspect of the present invention, a method of said compounds for treatment of cancer or tumor related diseases, which comprises administering to a subject in needs thereof an effective amount of said Compound A and B or a mixture thereof.

The structure of said Compound A is shown as follows:

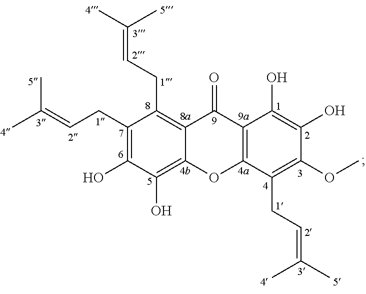 [SMILES: COc1c(O)c(O)c2c(=O)c3c(CC=C(C)C)c(CC=C(C)C)c(O)c(O)c3oc2c1CC=C(C)C]

The structure of said Compound B is shown as follows:

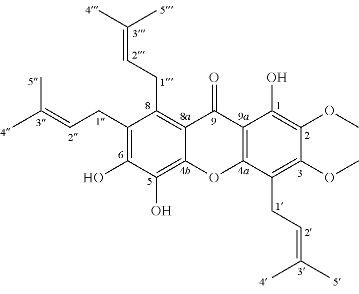 [SMILES: COc1c(OC)c(O)c2c(=O)c3c(CC=C(C)C)c(CC=C(C)C)c(O)c(O)c3oc2c1CC=C(C)C]

Those skilled in the art will appreciate that the present invention described herein is susceptible to variations and modifications other than those specifically described.

The present invention includes all such variation and modifications. The present invention also includes all of the steps and features referred to or indicated in the specification, individually or collectively, and any and all combinations or any two or more of the steps or features.

Throughout the present specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the present invention and apply throughout. Unless otherwise defined, all other technical terms used herein have the same meaning as commonly understood to one of ordinary skill in the art to which the invention belongs.

Other aspects and advantages of the present invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows the effect of compound A on a human immortalized non-tumorigenic liver cell line, namely MIHA.

FIG. 2 shows the key HMBC (H→C) correlations of Compound A and Compound B.

### DETAILED DESCRIPTION OF INVENTION

Method of Isolating Compounds from Source

The leaves of Garcinia nujiangensis are pulverized and extracted with acetone at room temperature three times, a week for each time. The acetone-soluble extracts are then suspended in hot water and extracted with CH₂Cl₂. The CH₂Cl₂ fraction is purified by column chromatography and HPLC to afford Compound A and B.

Separation

The CH₂Cl₂-soluble partition is subjected to a silica gel column (100-200 mesh, 1000 g), eluted with gradient CH₂Cl₂/MeOH (1:0, 0:1) to afford 8 fractions (Fr. A-H) according to TLC monitor. Fr. A is chromatographed on a column of silica gel eluted successively with a gradient of petroleum ether/ethyl acetate (3:1) to give four sub-fractions. (Fr. Ba-Fr. Bd). Fr. Bc is further separated by Octadesylsilane (ODS) with methyl alcohol/water (9:1) and purified by semipreparative HPLC (Agilent 1200 Series) with methyl alcohol/water (93:7 containing 0.1% Trifluoroacetic acid).

Compound Characterization

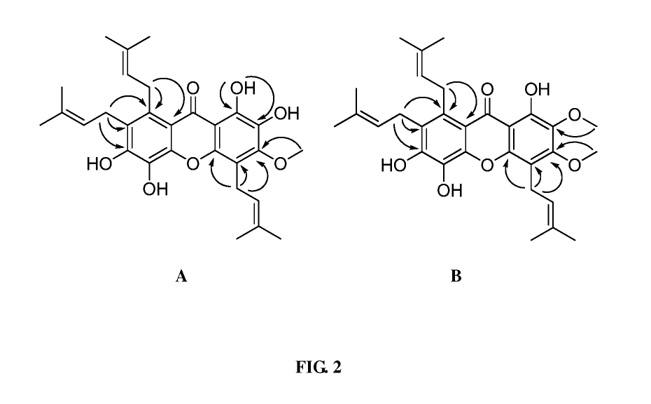

Compound A is shown to have the molecular formula C₂₉H₃₄O₇ by HRESIMS measurement (m/z 493.2161, [M-1]⁻). The IR spectrum exhibited strong bands due to the phenolic hydroxyl (3423 cm⁻¹) and chelated carbonyl (1639 cm⁻¹) group. The UV absorptions (MeOH) at λₘₐₓ 264.1 and 331.6 nm indicate that Compound A be a hydroxyl xanthone derivative. ¹H and ¹³C NMR data (Table 1), aided by HSQC experiments, disclose the presence of a carbonyl, 15 sp² quaternary carbons (seven of which are oxygen bearing), three sp² methine groups, three sp³ methylene groups, a methoxyl group and six methyl groups. The initial analysis of the NMR spectroscopic data of Compound A indicates that the compound consists of a xanthone skeleton with a methoxyl and three isoprenyl moieties. The ¹H NMR spectrum of Compound A reveals the proton signals of a methoxyl signal at δ_{H} 3.88 (3H, s) and three isoprenyl moieties, the first one of which has a pair of gem-dimethyl signals at δ_{H} 1.78 (3H, s, H-5′) and δ_{H} 1.61 (3H, s, H-4′), a methine signal at δ_{H} 5.30 (1H, t, J=7.2 Hz, H-2′), and a methylene signal at δ_{H} 3.57 (2H, d, J=7.2 Hz, H-1′); the second one has a pair of gem-dimethyl signals at δ_{H} 1.71 (3H, s, H-5″) and δ_{H} 1.63 (3H, s, H-4″), a methine signal at δ_{H} 4.99 (1H, m, H-2″), and a methylene signal at δ_{H} 3.41 (2H, d, J=6.0 Hz, H-1″); and the third one has a pair of gem-dimethyl signals at ω_{H} 1.63 (3H, s, H-5′″) and δ_{H} 1.71 (3H, s, H-4′″), a methine signal at δ_{H} 4.98 (1H, m, H-2′″), and a methylene signal at δ_{H} 3.98 (2H, d, J=4.8 Hz, H-1′″). The locations of three isoprenyl moieties are placed at the C-4 (δ_{C} 111.6), C-7 (δ_{C} 125.4), and C-8 (δ_{C} 133.6) positions based on the correlations (FIG. 2) in HMBC spectrum of Compound A. Furthermore, a chelated hydroxy proton at δ_{H} 13.40 (1H, s, 1-0H) shows a definite cross peak to the carbon signal at C-1 (δ_{C} 148.0) and C-2 (δ_{C} 133.0) (FIG. 2). The methoxy moiety located at C-3 (δ_{C} 153.0) is confirmed by HMBC on the correlation of OCH₃ (δᵤ 3.88)/C-3 (δ_{C} 153.0) (FIG. 2). After comparing the ¹H and ¹³C NMR data of A with those of known xanthone having the same partial structure, the substituted pattern of ring C is found to be similar to that of 1,3,5,6-tetrahydroxy-4,7,8-tri(3-methyl-2-butenyl)xanthone. Therefore, four hydroxyl groups are located at C-1 (δ_{C} 148.0), C-3 (δ_{C} 153.0), C-5 (δ_{C} 129.9), and C-6 (δ_{C} 151.4) by analysis of the HSQC and HMBC data, respectively. Thus, compound A is determined to be 1,2,5,6-tetrahydroxy-3-methoxy-4,7,8-tri(3-methylbut-2-enyl)-xanthone.

Compound B was shown to have the molecular formula C₃₀H₃₆O₇ by HRESIMS measurement (m/z 507.2394, [M-1]⁻). The IR spectrum exhibits strong bands due to the phenolic hydroxyl (3434 cm⁻¹) and chelated carbonyl (1648 cm⁻¹) groups. The UV absorptions (MeOH) at λₘₐₓ 245.7, 269.0 and 329.8 nm indicate that Compound B be a hydroxyl xanthone derivative. ¹H and ¹³C NMR data (Table 1), aided by HSQC experiments, disclose the presence of a carbonyl, 15 sp² quaternary carbons (seven of which were oxygen bearing), three sp² methine groups, three sp³ methylene groups, two methoxyl groups and six methyl groups. The initial analysis of the NMR spectroscopic data of Compound B indicates that the compound consists of a xanthone skeleton with three prenyl and two methoxyl moieties. The ¹H NMR spectrum of Compound B reveals the proton signals of three prenyl moieties, the first one of which has a pair of gem-dimethyl signals at δ_{H} 1.78 (3H, s, H-5′) and δ_{H} 1.61 (3H, s, H-4′), a methine signal at δ_{H} 5.30 (1H, t, J=7.2 Hz, H-2′), and a methylene signal at δ_{H} 3.56 (2H, m, H-1′); the second one of which has a pair of gem-dimethyl signals at δ_{H} 1.72 (3H, s, H-5″) and δ_{H} 1.62 (3H, s, H-4″), a methine signal at δ_{H} 5.01 (1H, m, H-2″), and a methylene signal at δ_{H} 3.32 (2H, m, H-1″); and the third one of which has a pair of gem-dimethyl signals at δ_{H} 1.62 (3H, s, H-5′″) and δ_{H} 1.72 (3H, s, H-4′″), a methine signal at δ_{H} 4.98 (1H, m, H-2′″), and a methylene signal at δ_{H} 3.95 (2H, m, H-1′″). The locations of three prenyl moieties are placed at the C-4 (δ_{C} 111.5), C-7 (δ_{C} 125.7), and C-8 (δ_{C} 133.7) positions based on the correlations of H-1′ (δ_{H} 3.56)/C-3 (δ_{C} 157.2), H-1′ (δ_{H} 3.56)/C-4 (δ_{C} 111.5), and H-1′ (δ_{H} 3.56)/C-4-a (δ_{C} 148.1); H-1″ (δ_{H} 3.32)/C-6 (δ_{C} 153.3), H-1″ (δ_{H} 3.32)/C-7 (δ_{C} 125.7) and H-1″ (δ_{H} 3.56)/C-8 (δ_{C} 133.7); and H-1′″ (δ_{H} 3.95)/C-7 (δ_{C} 125.7), H-1′″ (δ_{H} 3.56)/C-8 (δ_{C} 133.7) and H-1′″ (δ_{H} 3.55)/C-8a (δ_{C} 109.8) (FIG. 2) in HMBC spectrum of Compound B, respectively. Two methoxy groups are located at C-2 (135.1) and C-3 (δ_{C} 157.2) confirmed by HMBC on the correlations of OH₃ (δ_{H} 3.80)/C-2 (δ_{C} 135.1) and (δ_{H} 3.95)/C-2 (δ_{C} 157.2) (FIG. 2), Comparing the ¹H and ¹³C NMR data of Compound B with those of the known xanthones having the same partial structure, the substituted pattern of ring C is similar to that of 1,3,5,6-tetrahydroxy-4,7,8-tri(3-methyl-2-butenyl)xanthone. Thus, Compound B is determined to be 1,2,5,6-tetrahydroxy-2,3-dimethoxy-4,7,8-tri(3-methylbut-2-enyl)-xanthone.

TABLE 1
¹H and ¹³C NMR Spectroscopic Data
of Compound A and B in DMSO-d₆

| | Compound A | | Compound B | |
|---|---|---|---|---|
| position | δ_{C} | δ_{H} (J in Hz) | δ_{C} | δ_{H} (J in Hz) |
| 1 | 148.0 | | 147.4 | |
| 2 | 133.0 | | 135.1 | |
| 3 | 153.0 | | 157.2 | |
| 4 | 111.6 | | 111.5 | |
| 4a | 145.2 | | 148.1 | |
| 4b | 147.6 | | obscured | |
| 5 | 129.9 | | 130.0 | |
| 6 | 151.4 | | 153.3 | |
| 7 | 125.4 | | 125.7 | |
| 8 | 133.6 | | 133.7 | |
| 8a | 110.2 | | 111.5 | |
| 9 | 183.1 | | 183.1 | |
| 9a | 105.1 | | 105.2 | |
| 1′ | 21.9 | 3.57, d (7.2) | 21.8 | 3.56, m |
| 2′ | 123.4 | 5.20, d (7.2) | 123.1 | 5.20, m |
| 3′ | 130.7 | | 130.0 | |
| 4′ | 17.9 | 1.78, s | 17.9 | 1.78, s |
| 5′ | 25.7 | 1.61, s | 25.7 | 1.61, s |
| 1″ | 24.5 | 3.34, m | 24.5 | 3.32, m |
| 2″ | 123.1 | 4.98, overlap | 123.1 | 5.01, overlap |
| 3″ | 131.0 | | 130.1 | |
| 4″ | 18.1 | 1.71, s | 18.2 | 1.72, s |
| 5″ | 25.7 | 1.63, s | 25.8 | 1.62, s |
| 1″′ | 28.3 | 3.98, d (4.8) | 28.3 | 3.95, m |
| 2″′ | 125.4 | 4.99, overlap | 124.4 | 4.98, overlap |
| 3″′ | 129.9 | | 130.1 | |
| 4″′ | 18.2 | 1.71, s | 18.1 | 1.72, s |
| 5″′ | 25.7 | 1.63, s | 25.7 | 1.62, s |
| 1-OH | | 13.40, brs | | 13.67, brs |
| 3-OCH₃ | 60.4 | 3.88, s | 60.4 | 3.80, s |
| 2-OCH₃ | | | 61.2 | 3.95, s |

Data were recorded with a Bruker DRX-400 MHz spectrometer, chemical shifts (d) are expressed in ppm, J in Hz; assignments were confirmed by HSQC and HMBC.

### Example

### The Preparation of Compound A and B

The leaves of Garcinia nujiangensis are pulverized and extracted with acetone at room temperature three times, a week for each time. The acetone-soluble extract is suspended in hot water and extracted with CH₂Cl₂, the CH₂Cl₂-soluble fraction is subjected to a silica gel column (100-200 mesh, 1000 g), eluted with gradient CH₂Cl₂/MeOH (1:0, 0:1) to afford seven fractions (Fr.A-G) according to TLC monitor. Fr. A is chromatographed on a column of silica gel eluted successively with a gradient of petroleum ether/ethyl acetate (3:1) to give four sub-fractions. (Fr.Ba-Fr.Bd). Fr.Bc is further separated by ODS with methyl alcohol/water (9:1) and purified by semipreparative HPLC (Agilent 1200 Series) with methyl alcohol/water (93:7 containing 0.1% Trifluoroacetic acid).

Cytotoxicity

The cytotoxicity of Compound A to normal liver cells immortalized MIHA is tested with MTT assay.

Procedure

For MTT assay, 10 μL MTT solution is added into each well of a 96-well plate. After 2 h incubation at 37° C., 100 μL 10% SDS solution with 10 mM HCl is added to dissolve the purple crystals. After 24 h incubation, the optical density (OD) readings at 595 nm are measured using a Perkin-Elmer Victor plate reader. The values of IC₅₀ are determined using the method described in the cited literatures with some modifications. To determine the IC₅₀ value of a compound in a cell line, the growth inhibitory effect of Compound A at different concentrations is measured by MTT assay. For each tested concentration, 3000 cells suspended in 100 μL culture medium are seeded in each well of a 96-well plate. The well containing 100 μL culture medium without cells is used as a blank. After an overnight culture, Compound A at the designed concentration is added. The OD values of the control group at 0 h (C₀) and 72 h (C₇₂) together with the compound treated groups at 72 h (T₇₂) from the MTT assay are measured using the Perkin-Elmer Victor plate reader. The survival rates are calculated using the formula (T₇₂−C₀)/(C₇₂−C₀)×100%. The inhibition rates are calculated using the following formula [1−(T₇₂−C₀)/(C₇₂−C₀)]×100% and plotted against logarithmic concentrations by sigmoidal fit. IC₅₀ is defined as the concentration of a compound inhibiting 50% of cell growth. Each IC₅₀ test is performed in triplicate and repeated at least three times. The results showed in Table 2.

TABLE 2
the Effect of Compound A on Normal
Liver Cells Immortalized MIHA

| Concentration of | | Survival rate |
|---|---|---|
| compound A | OD value | (%) |
| 40 μg/mL | 1.5405 | 68.01325 |
| 20 μg/mL | 1.9455 | 85.89404 |
| 10 μg/mL | 1.9355 | 85.45254 |
| 5 μg/mL | 2.3055 | 101.7881 |
| control | 2.265667 | — |

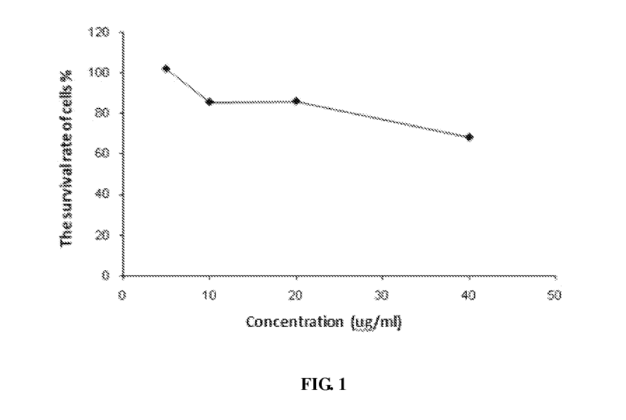

As shown in FIG. 1, the curve with a survival rate of compound A on MIHA cell half toxic concentration for TC₅₀>40 μg/mL, compound A shows low toxicity on normal cells.

Anti-Cancer Biological Activity of Compound A and Compound B

All test samples are dissolved in dimethyl sulfoxide (DMSO) to make stock solutions and further diluted in culture medium upon assay. Human cancer cell lines including U87, MDA-MB-231, AGS and HepG2 are cultured in RPMI 1640 or DMEM or DMEM/F12 medium, containing 10% fetal bovine serum. All cell lines are maintained at 37° C. in a humidified environment containing 5% CO₂. To determine the effects of the compounds on cell viability, cell number is quantified using a standard colorimetric MTT assay. Cells are placed in a 96-well plate (5×10³ cells/well) and allowed to attach overnight. Cells are treated with 5, 10, 20, 40 μM of each compound in culture medium for 72 h, respectively. Then, culture media are added with 20 μL of MTT (5 mg/mL stock in PBS) per well and incubated for 4 h at 37° C. Finally, culture media are discarded and 150 μL of DMSO is added to each well to dissolve the purple formazan crystals. Absorbance of the solution is measured using microplate reader spectrophotometer (Bio-Rad Laboratories, Inc., Hercules, Calif.), at a wavelength of 490 nm. Absorbance of untreated cells in medium (negative control) is 100%. Curcumin and paclitaxel are used as the positive control. Table 3 shows the effect of compound A on inhibiting HepG2 cell growth. Table 4 shows the IC₅₀ of Compound B against the four cancer cell lines. The data indicates that compound B has cytotoxic effects on these cancer cell lines. Therefore, compound A and B of the present invention can be developed as anticancer drugs.

TABLE 3
The effect of compound A on inhibiting HepG2

| | | |
|---|---|---|
| Concentration of | | |
| compound A | OD value | Inhibition rate % |

| | | | |
|---|---|---|---|
| 100 | μg/mL | 0.6285 | 74.2629 |
| 50 | μg/mL | 0.7495 | 69.30794 |
| 25 | μg/mL | 0.5715 | 76.59705 |
| 12.5 | μg/mL | 0.5955 | 75.61425 |
| 6.25 | μg/mL | 0.996 | 59.21376 |
| 3.125 | μg/mL | 2.075667 | 15.00137 |
| 1.6 | μg/mL | 2.242667 | 8.162708 |
| 0.8 | μg/mL | 2.251667 | 7.794158 |

| | | |
|---|---|---|
| Cell control | 2.442 | — |

TABLE 4
Cytotoxicity Data IC₅₀ (μM) of
Compound B against four Tumor Cells

| Cell Line | U87 | MDA-MB-231 | AGS | HepG2 |
|---|---|---|---|---|
| Compound B | 55.31 | 70.54 | 31.13 | 20.27 |
| Curcumin | 20.81 | 3.76 | 4.38 | Not test |
| Paclitaxel | 285.4 | — | 23.11 | Not test |

In summary, our results indicated that compound A had a strong effect on HepG2 cells growth with IC₅₀ at 5.3 μg/mL concentration. Compound B has cytotoxic effects on four cancer cell lines. Therefore, compound A and B can be used for potential anticancer drugs.

Plant Material

The leaves of G. nujiangensis were collected in Nujiang, Yunnan Province, People's Republic of China, in August 2010. The plant material was identified by Prof. Yuanchuan Zhou, T. C. M. of Yunnan. A voucher sample (G. N. 0001) was deposited in the innovative medicine laboratory, T. C. M of Shanghai.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

### INDUSTRIAL APPLICABILITY

The compounds of the present invention, namely compound A [1,2,5,6-tetrahydroxy-3-methoxy-4,7,8-tri(3-methylbut-2-enyl)-xanthone] and compound B [1,2,5,6-tetrahydroxy-2,3-dimethoxy-4,7,8-tri(3-methylbut-2-enyl)-xanthone] isolated from Garcinia nujiangensis is useful in the preparation of a pharmaceutical composition for treating cancer or tumor related diseases.

What we claim:

1. A composition for treating cancer or tumor related diseases in liver comprising an effective amount of xanthone derivatives having a formula of B as follows:

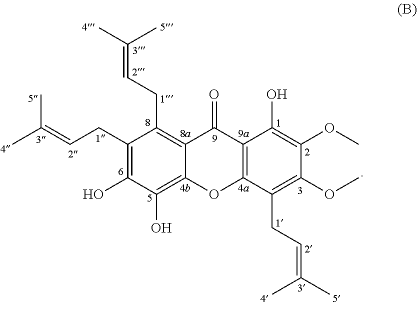 [SMILES: COc1c(OC)c(O)c2c(=O)c3c(CC=C(C)C)c(CC=C(C)C)c(O)c(O)c3oc2c1CC=C(C)C]

2. The composition of claim 1, wherein said xanthone derivatives having said formula B is 1,2,5,6-tetrahydroxy-2,3-dimethoxy-4,7,8-tri(3-methylbut-2-enyl)-xanthone.

3. The composition of claim 1 is administered to a subject in needs thereof as anti-cancer/anti-tumor drugs or treatments.

4. The composition of claim 1, wherein said xanthone derivatives having said formula B are extracted and separated from a natural source.

5. The composition of claim 4, wherein the natural source is a herb.

6. The composition of claim 5, wherein the herb is Garcinia nujiangensis.

7. The composition of claim 1 for treating cancer or tumor related disease in liver is prepared by
a) pulverizing leaves of Garcinia nujiangensis followed by extraction with acetone at room temperature for at least one time to yield an acetone-soluble extract;
b) suspending the acetone-soluble extract in hot water followed by extraction with CH₂Cl₂ to yield a CH₂Cl₂-soluble fraction;
c) subjecting the CH₂Cl₂-soluble fraction to a silica gel column followed by elution with a gradient of CH₂Cl₂/MeOH (1:0, 0:1) to afford at least seven fractions according to TLC monitor;
d) a first fraction of the at least seven fractions collected from (c) is chromatographed on a column of silica gel eluted successively with a gradient of petroleum ether/ethyl acetate (3:1) to give at least four sub-fractions;
e) one of the at least four sub-fractions is further separated by octadesylsilane with methyl alcohol/water (9:1) and purified by semipreparative HPLC with methyl alcohol/water (93:7) which contains 0.1% Trifluoroacetic acid.

8. The composition of claim 7, wherein said extraction in (a) is performed at least once a week for at least three consecutive weeks.

9. The composition of claim 7, wherein said silica gel column used in (c) is 100-200 mesh in 1000 g.
